(19) United States
(12) Patent Application Publication
(10) Pub. No.: US 2006/0264851 A1
(43) Pub. Date: Nov. 23, 2006

(54) Feminine hygiene douche

(76) Inventor: India Coleman, Inglewood, CA (US)

Correspondence Address: Raymond Y. Chan, 108 N. Ynez Ave., #128, Monterey Park, CA, 91754, US

(21) Appl. No.: 11/125,973

(22) Filed: May 9, 2005

(51) Int. Cl. A61M 31/00

(52) U.S. Cl. 604/279; 128/838

(57) ABSTRACT

A hygiene douche device includes a douche body having an opening and a solution cavity for containing cleansing solution therein; and brush head, which is sized and shaped adapted for slidably inserting into a vagina. The brush head includes a tubular guiding arm extended from the opening of the douche body. The guiding arm has a head portion, a dispensing channel communicating with the solution cavity, and a plurality of dispensing pores provided on the head portion to communicate with the dispensing channel and a brush member provided at the head portion of the guiding arm and arranged in such a manner that when the brush member is inserted into the vagina. The brush member is adapted for rubbing on an inner wall of the female vagina for administering the cleansing solution thereto.

### BACKGROUND OF THE PRESENT INVENTION

1. Field of Invention

The present invention relates to feminine hygiene douche, and more particularly relates to a feminine hygiene douche which includes a brush head applicable to the vagina of a body for easy and effective cleaning.

2. Description of Related Arts

Vaginal douches are essential for maintaining the personal hygiene of women as poor hygiene may lead to infection or inflammation of the vagina or its vicinities. Also, it is very important to prevent any unpleasant smell or odor resulted from secretions from the lining of vagina and poor personal hygiene. The area of vagina and its vicinities is very delicate and sensitive while the unique angle of curvature of the passage of vagina induces difficulties of reaching and cleaning. Therefore, a vaginal douche of substantive flexibility and rigidity is required for facilitating the insertion of the douche device while not causing any damage to the lining of the vagina. A stiff body will not be able to fit the specific angle of the passage of vagina while a soft body will not have enough rigidity supporting the insertion of the douche or application of the douche.

Conventionally, vaginal douches are designed for applying a douche liquid to the vaginal of a body for cleaning. Due to the difficulties of application of the douche into the vagina, an applicator is commonly used as an accessory kit assisting the insertion of the douche into the vagina such that a douche liquid is capable of administering into the lining of the vagina through the opening on the douche for achieving the cleaning purposes. The use of the applicator for insertion of the douche complicates the application of the douche and makes it inconvenience for daily use.

On the other hand, the mechanism of the cleaning is usually based on the flowing of the douche liquid passing through the vagina or the absorption of the douche liquid by the lining of vaginal douche. The distribution and flow rate of the douche liquid is difficult to control and an uneven distribution of the douche liquid is resulted. Thus, the poor control of douche liquid distribution substantively adversely affects the cleaning of the vaginal area or its vicinities.

The spilling of the douche liquid due to poor handling is another problem of the conventional douche device. Since the douche liquid cannot be handled easily, spilling of the douche liquid is not uncommon. The spill to the clothing of a user is undesirable and may lead to embarrassing circumstances. Thus, the use of the douche is not commonly accepted for daily uses or carrying out as a daily hygiene device in the handbags of women.

The conventional douche device usually has a container for storing the equipped douche liquid. The constraint of the shape of the douche device for fitting the vaginal area resulted in a generally elongated douche device with a certain length, and it is absolutely not convenience for carrying for daily use.

### SUMMARY OF THE PRESENT INVENTION

A main object of the present invention is to provide a hygiene douche applicable to fit the vaginal area with effective cleaning effect without the use of applicator and to promote the use of the hygiene douche for daily use.

Another object of the present invention is to provide a hygiene douche which is simple in construction, easy and convenience to handle, while being capable of providing a highly effective cleaning, especially suitable for daily use.

Another object of the present invention is to provide a hygiene douche comprising a douche body having a plurality of dispensing pores connecting to a dispensing channel adapted for attaching to a douche body containing cleansing solution such that the cleansing solution is adapted to be passing through the dispensing channel to the dispensing pores for application.

Another object of the present invention is to provide a hygiene douche comprising a douche body with a brush head for effective cleansing through brushing and that the brush head is capable of controlling a flow rate of the cleansing solution and hence reducing the chance of spilling of the cleansing solution.

Another object of the present invention is to provide a hygiene douche comprising a douche body with a brush head made of a predetermined material for brushing action and hence enhancing the cleaning effect without causing any damage to a user.

Another object of the present invention is to provide a hygiene douche comprising a douche body adapted for attaching to a brush head such that different kinds of cleansing solution may be selected and contained in the douche body according to the preference of a user.

Another object of the present invention is to provide a hygiene douche comprising a douche body detachably attached to a brush head such that the length of the is hygiene douche body may be shortened.

Another object of the present invention is to provide a hygiene douche comprising a douche body attached to a brush head, wherein the douche body is made of elastic material adapted for reducing in volume corresponding to the reduced volume of cleansing solution such that a constant pressure is maintained and that the cleansing solution is capable of forcing out though the opening to the brush head.

In order to achieve the above objects, the present invention provides a hygiene douche comprising:

a brush head having an upper portion and a lower portion with a predetermined flexibility, generally of a tubular and elongated shape, and an inner passage provided therein passing through the upper portion and the lower portion of the brush head, wherein the upper portion of the brush head has a plurality of pores leading from the inner passage and the lower portion of the brush head has an opening and a connecting portion, comprising:

a brush member provided at an upper portion of the brush head made of a predetermined material having a predetermined density such that a liquid is capable of passing through the pores to the brush member for douching; and

a douche body having an attaching portion detachably connected to the connecting portion of the lower portion of the brush head enclosed the opening of the lower portion of the brush head adapted for containing a douche liquid.

These and other objectives, features, and advantages of the present invention will become apparent from the following detailed description, the accompanying drawings, and the appended claims.

### BRIEF DESCRIPTION OF THE DRAWINGS

FIG. 1 is the perspective view of the feminine hygiene douche according to the preferred embodiment of the present invention.

FIGS. 2A to 2C are the perspective view of brush heads of different materials of the feminine hygiene douche according to the preferred embodiment of the present invention.

FIG. 3 is the partial sectional view of brush head of the feminine hygiene douche according to the preferred embodiment of the present invention

### DETAILED DESCRIPTION OF THE PREFERRED EMBODIMENT

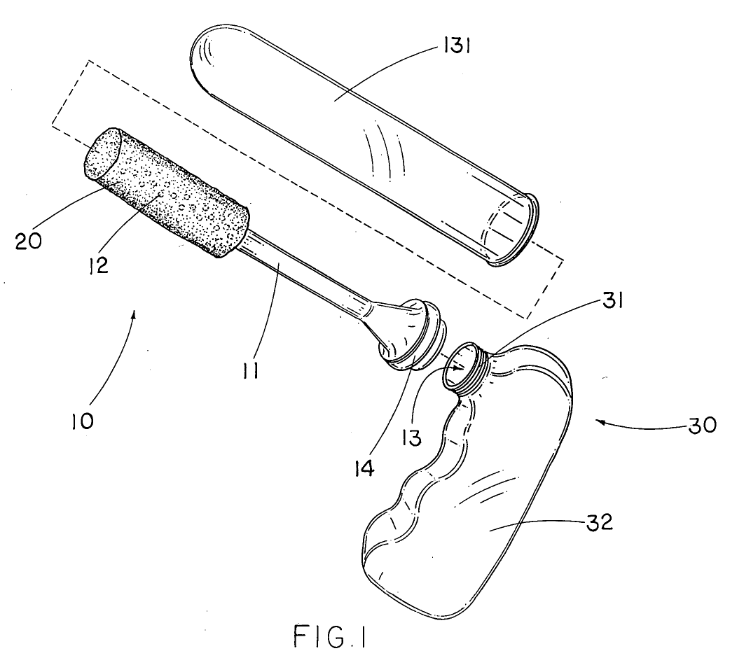

Referring to FIG. 1 of the drawings, a feminine hygiene douche of the present invention is illustrated. The hygiene douche comprises a brush head 10 and a douche body 30. The douche body 30 has an opening 31 and a solution cavity 32 for containing cleansing solution therein.

The brush head 10 is generally of tubular and elongated shape and has a predetermined flexibility for facilitating the insertion of the douche body into the vagina of a body for douching. The brush head 10 is sized and shaped for slidably inserting into a vagina. The brush head 10 comprises a tubular guiding arm 11 extended from the opening of the douche body, wherein the guiding arm 11 has a head portion 15, a dispensing channel 13 communicating with the solution cavity, and a plurality of dispensing pores 12 provided on the head portion 20 to communicate with the dispensing channel 13. The brush head 10 also comprises a brush member 20 provided at the head portion of the guiding arm and arranged in such a manner that when the brush member is inserted into a vagina, the brush member is adapted for rubbing on an inner wall of the vagina for administering the cleansing solution thereto

The guiding arm 11 has a detachable tail portion 14 detachably fastening with the opening of the douche body for filling or refilling the cleansing solution in the solution cavity when the brush head is detached from said douche body.

Preferably, a sectional area of the dispensing channel near a portion of the brush head closer to the tail portion is larger than a sectional area of the dispensing channel closer to the brush head, and that the sectional area of the dispensing channel decreases gradually from the tail portion towards the portion closer to the brush member such that the pressure at the tail portion is lowered and the inner pressure at the brush member is higher through capillary action, and that any cleansing solution from the tail portion is capable of forcing upwards towards the brush member.

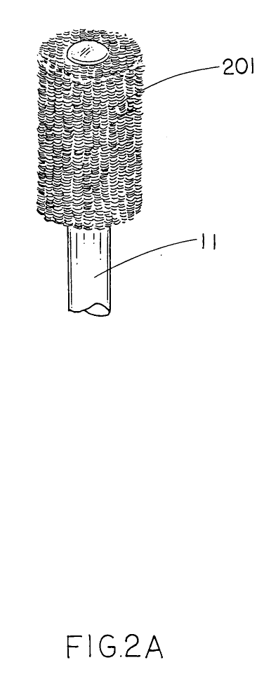

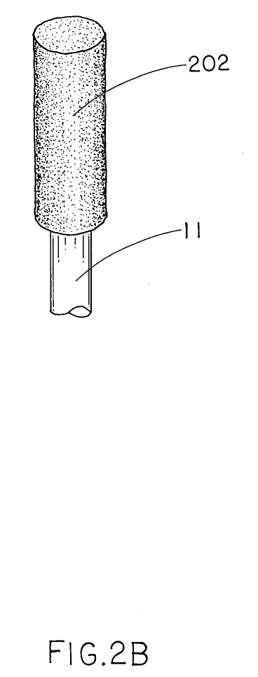

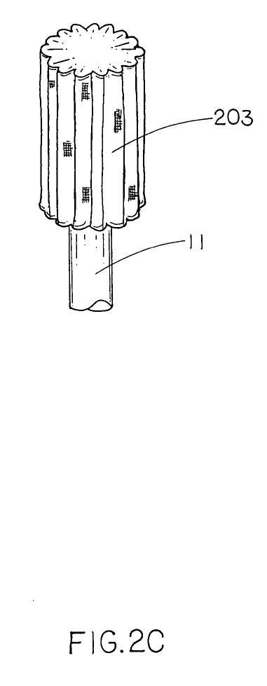

The brush member 20 is provided at the head portion of the guiding arm. Referring to FIGS. 2A to 2C of the drawings, the brush member 20 may be made of bristle, foam or cloth according to the preferred embodiment of the present invention. In fact, the brush member 20 is preferably made of a material having a predetermined density and softness such that the density is low enough for allowing passage of liquid through the pores 12 of the brush head to the brush member 20 and the softness is sufficient to provide a comfortable feeling to a user in using the hygiene douche of the present invention such that rubbing action is allowed.

The choices of bristle, foam or cloth are designed for different uses. For instance, foam and cloth are suitable for users of delicate sensitivity while bristle is suitable for users of medium sensitivity.

Rotational movement of the brush member 20 allows swapping, brushing and rubbing action for the lining of the vaginal area for enhancing the cleaning effect. Furthermore, a continuously administration of cleansing solution through the dispensing pores is possible which further provides lubricating and cleaning effects simultaneously.

The brush member 20 provided on the upper portion of the douche body induces a difference in thickness between the head and tail portion of the guiding arm such that when the brush head is put inside a vaginal area, an area is defined between the brush head and the vaginal area that the chance of direct contact between the brush head and the vaginal area can be lowered. The area defined serves as a comfort zone for the longitudinal movement of the douche body.

The douche body may have a deformable squeezing portion adapted to be compressed to generate a pumping effect of the douche body for discharging the cleansing solution in the solution cavity to the brush member through the dispensing channel.

Referring to FIG. 2A of the drawings, the brush member comprises a plurality of bristles radially extended from the head portion of the guiding member for being soaked with the cleansing solution to gently rub on said inner wall of the vagina.

Referring to FIG. 2B of the drawings, the brush member comprises a foaming element coaxially encircling around the head portion of the guiding member for being soaked with the cleansing solution to gently rub on the inner wall of the vagina.

Referring to FIG. 2C of the drawings, the brush member comprises a clothing element coaxially surrounding the head portion of the guiding member for being soaked with the cleansing solution to mildly swapping on the inner wall of the vagina.

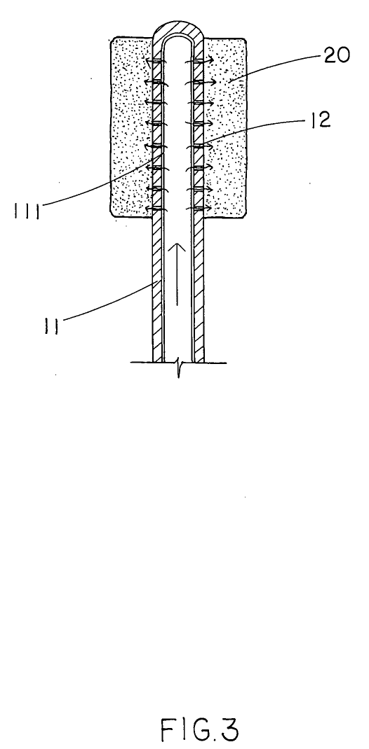

Referring to FIG. 3 of the drawings, the guiding arm further comprises a diffusion membrane 111 formed on an inner wall of the guiding arm at the head portion thereof for controllably diffusing the cleansing solution from the dispensing channel to the brush member through the dispensing pores so as to prevent a backflow of the cleansing solution. The flow rate of the cleansing solution may be reduced at a controllable level for a user.

The plurality of dispensing pores provided at the head portion of the guiding arm facilitate the delivering of cleansing solution to the brush member 20. In other words, any cleansing solution used together with the feminine hygiene douche of the present invention is delivered into the vaginal area through the dispensing pores.

The diffusion membrane 111 is made of materials permeable to liquid and preferably having a certain level of water retainable capacity such as cloth and cotton. Thus, the flow rate of the cleansing solution can be controlled more easily and the spilling the cleansing solution during the use of the hygiene douche is avoided. The use of the diffusion membrane as an intermediate layer greatly eliminates the possibility of spillage and associated embarrassing situation and thus assisting the promotion of use of the hygiene douche as a daily portable douche in handbags of females.

Referring to FIG. 1 of the drawings, the tail portion 14 has a bored hole with inner thread portion and the douche body has an outer thread portion for connecting the brush head and the douche body. Alternately, the tail portion and the area surrounding the opening of the douche body may be another design of coupling means such as coupling devices.

The douche body 30 may further have a contour body for enhancing the pumping action while serving as a handle, as shown in FIG. 1 of the drawings. Since the douche body is capable of detachably connected to the brush head 10, the length of the brush head equipped with the douche body may be reduced for the ease for carriage. A cap cover 131 for fittingly detachably attached to the brush head 20 may also be included for covering the brush head for hygiene purpose. The cap cover 131 preferably has an elongated shape for sealedly enclosing said brush head therein. The tail portion 14 may further has a protruded rim corresponding to an opening of the cap cover 131 for fittingly locking the cap cover 131.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A hygiene douche device, comprising:
a douche body having an opening and a solution cavity for containing cleansing solution therein; and
a brush head, which is sized and shaped adapted for slidably inserting into a female vagina, comprising:
a tubular guiding arm extended from said opening of said douche body, wherein said guiding arm has a head portion, a dispensing channel communicating with said solution cavity, and a plurality of dispensing pores provided on said head portion to communicate with said dispensing channel; and
a brush member provided at said head portion of said guiding arm and arranged in such a manner that when said brush member is inserted into said female vagina, said brush member is adapted for rubbing on an inner wall of said female vagina for administering said cleansing solution thereto.

2. The hygiene douche device, as recited in claim 1, wherein said guiding arm has a detachable tail portion detachably fastening with said opening of said douche body for refilling said cleansing solution in said solution cavity when said brush head is detached from said douche body.

3. The hygiene device, as recited in claim 1, wherein said douche body has a deformable squeezing portion adapted to be compressed to generate a pumping effect of said douche body for discharging said cleansing solution in said solution cavity to said brush member through said dispensing channel.

4. The hygiene device, as recited in claim 2, wherein said douche body has a deformable squeezing portion adapted to be compressed to generate a pumping effect of said douche body for discharging said cleansing solution in said solution cavity to said brush member through said dispensing channel.

5. The hygiene device, as recited in claim 1, wherein said brush member comprises a plurality of bristles radially extended from said head portion of said guiding member for being soaked with said cleansing solution to gently rub on said inner wall of said female vagina.

6. The hygiene device, as recited in claim 2, wherein said brush member comprises a plurality of bristles radially extended from said head portion of said guiding member for being soaked with said cleansing solution to gently rub on said inner wall of said female vagina.

7. The hygiene device, as recited in claim 3, wherein said brush member comprises a plurality of bristles radially extended from said head portion of said guiding member for being soaked with said cleansing solution to gently rub on said inner wall of said female vagina.

8. The hygiene device, as recited in claim 4, wherein said brush member comprises a plurality of bristles radially extended from said head portion of said guiding member for being soaked with said cleansing solution to gently rub on said inner wall of said female vagina.

9. The hygiene device, as recited in claim 1, wherein said brush member comprises a foaming element coaxially encircling around said head portion of said guiding member for being soaked with said cleansing solution to gently rub on said inner wall of said female vagina.

10. The hygiene device, as recited in claim 2, wherein said brush member comprises a foaming element coaxially encircling around said head portion of said guiding member for being soaked with said cleansing solution to gently rub on said inner wall of said female vagina.

11. The hygiene device, as recited in claim 3, wherein said brush member comprises a foaming element coaxially encircling around said head portion of said guiding member for being soaked with said cleansing solution to gently rub on said inner wall of said female vagina.

12. The hygiene device, as recited in claim 4, wherein said brush member comprises a foaming element coaxially encircling around said head portion of said guiding member for being soaked with said cleansing solution to gently rub on said inner wall of said female vagina.

13. The hygiene device, as recited in claim 1, further comprising a diffusion membrane formed on an inner wall of said guiding arm at said head portion thereof for controllably diffusing said cleansing solution from said dispensing channel to said brush member through said dispensing pores so as to prevent a backflow of said cleansing solution.

14. The hygiene device, as recited in claim 4, further comprising a diffusion membrane formed on an inner wall of said guiding arm at said head portion thereof for controllably diffusing said cleansing solution from said dispensing channel to said brush member through said dispensing pores so as to prevent a backflow of said cleansing solution.

15. The hygiene device, as recited in claim 8, further comprising a diffusion membrane formed on an inner wall of said guiding arm at said head portion thereof for controllably diffusing said cleansing solution from said dispensing channel to said brush member through said dispensing pores so as to prevent a backflow of said cleansing solution.

16. The hygiene device, as recited in claim 12, further comprising a diffusion membrane formed on an inner wall of said guiding arm at said head portion thereof for controllably diffusing said cleansing solution from said dispensing channel to said brush member through said dispensing pores so as to prevent a backflow of said cleansing solution.

17. The hygiene device, as recited in claim 8, further comprising an elongated cap cover for sealedly enclosing said brush head therein.

18. The hygiene device, as recited in claim 12, further comprising an elongated cap cover for sealedly enclosing said brush head therein.

19. The hygiene device, as recited in claim 15, further comprising an elongated cap cover for sealedly enclosing said brush head therein.

20. The hygiene device, as recited in claim 16, further comprising an elongated cap cover for sealedly enclosing said brush head therein.
